(19) United States
(12) Patent Application Publication
(10) Pub. No.: US 2006/0095978 A1
(43) Pub. Date: May 4, 2006

(54) Screening assay

(76) Inventors: Christoph Burkhart, Vienna (AT); Ekke Liehl, Hinterbruhl (AT); Peter Mayer, Vienna (AT)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/529,166

(22) PCT Filed: Sep. 25, 2003
(86) PCT No.: PCT/EP03/10700

§ 371 Date: Aug. 8, 2005

Related U.S. Application Data
(60) Provisional application No. 60/413,706, filed on Sep. 26, 2002.
(60) Provisional application No. 60/413,702, filed on Sep. 26, 2002.
(60) Provisional application No. 60/413,703, filed on Sep. 26, 2002.

(51) Int. Cl. A01K 67/027

(52) U.S. Cl. 800/3; 800/18

(57) ABSTRACT

An in vivo screening assay for identifying an agent that interferes with T cell activation and/or -differentiation and/or modulation of other inflammatory effector cells.

The present invention relates to a screening assay, e.g. a method for identifying an agent that interferes with T cell activation and/or -differentiation and/or modulation of other inflammatory effector cells.

T cell receptors (TCRs) play a major role in the recognition of antigens and the subsequent immune response. Cytotoxic CD8⁺ T lymphocytes recognize antigens as peptide fragments in association with MHC class I molecules on the cell surface, whereas CD4⁺ T lymphocytes recognize antigens as peptide fragments in association with MHC class II molecules on the cell surface. The specific recognition induces a series of events including clonal expansion and the modulation (activation) of surface adhesion molecules such as, e.g. CD69, CD25, CD44, CD62, CD71 and CD11a in case of CD8⁺ T cells. Ultimately, in CD8⁺ T cells such changes may lead to cell differentiation into cytotoxic effector cells (=Th 1 cells), whereas in CD4⁺ T cells differentiation into T helper cells (=Th 2 cells) occurs, both capable of promoting T cell dependent immunity.

Activation of T cells may be assayed by determining either the amount of certain cell surface molecules or the presence of such molecules, which are up-regulated upon activation, such as e.g. CD25 and CD69 on peripheral blood lymphocytes and spleen cells in case of CD8⁺ T cells, or by measuring the amount of T-cell mediators (lymphokines), which are produced (released) upon activation. These mediators are able to activate or prime other cells, e.g. monocyte/macrophages, for enhanced release of pro-inflammatory and anti-inflammatory cytokines such as IL-1 α, IL-1 β, IL-6, IL-10 or TNF-α, respectively.

Cytokines may induce a complex spectrum of biological responses that are generally thought to regulate host defense. A subset of cytokines, including e.g. interleukin-1 (IL-1α and IL-1β) and tumor necrosis factor (TNF-α), mediate biological actions consistent with a role as inflammatory mediators. The temporary or sustained release of these pro-inflammatory cytokines plays a critical role in the pathogenesis of acute and chronic inflammation in several diseases e.g. autoimmune diseases such as rheumatoid arthritis, inflammatory bowel disease, systemic Lupus, psoriasis, multiple sclerosis and others.

But determining the amount of such T-cell and/or inflammatory effector cell derived mediators in vivo is difficult.

E.g. in normal mice the frequency of cells responding to a given antigen is usually below 1 in 100,000 lymphocytes and cytokine levels and/or levels of other mediators in serum are below the detection limit of assays. For testing potential drugs for their T-cell and/or other inflammatory effector cell activity in vivo, the use of an animal model in which levels of the mediators of interest can be determined in serum would be advantageous.

We have now surprisingly found a convenient possibility for the determination of T-cell and/or inflammatory effector cell activity, e.g. T-cell and/or inflammatory effector cell derived mediators, directly in vivo in serum.

In one aspect the present invention provides an kit (assay) for the determination of T-cell and/or inflammatory effector cell derived mediators directly in vivo in serum, comprising a mouse wherein the majority of T cells express a transgenic MHC class I restricted or MHC class II restricted T cell receptor.

T-cell and inflammatory effector cell derived mediators, such as mediators induced downstream of the lymphokine production, include e.g. cytokines, chemokines and/or other mediators, which are produced (released) upon T-cell and inflammatory effector cell activation, preferably cytokines and chemokines, such as selected from the group consisting of IL1-α, IL-1β, IL-2, IL-4, IL-6, IL-10, TNF-α and IFN-γ.

Mice wherein the majority of T cells express a transgenic MHC class I restricted or MHC class II restricted T cell receptor, such as transgenic mice expressing an ovalbumin (OVA-peptide)-specific TCR, e.g. include DO11.10 mice or OT-1 mice. OT-1 mice may be obtained e.g. as described in Hogquist et al., 1994, Cell 76(1)17-27, DO11.10 mice may be obtained e.g. as described in Murphy K. M. et al. 1990, Science 250:1720-1722.

In an OT-1 mouse with the specificity for a MHC class I restricted peptide, the MHC class I restricted peptide is preferably a (restricted) ovalbumin(OVA)-peptide, e.g. OVA₂₅₇₋₂₆₄. In a DO11.10 mouse with a specificity for a MHC class II restricted peptide, the MHC class II restricted peptide is preferably a (restricted) ovalbumin peptide, e.g. OVA₃₂₃₋₃₃₉.

In more detail, we have found that transgenic mice expressing an ovalbumin (OVA)-specific transgenic TCR, e.g. DO11.10 mice or OT-1 mice, and thus containing a high percentage of OVA-specific T cells e.g. in their lymphoid tissue, after stimulation with an appropriate stimulation agent, e.g. by administration of an antigen to the ovalbumin (OVA-peptide)—specific TCR, such as the OVA₃₂₃₋₃₃₉ peptide in DO 11.10 mice or the OVA₂₅₇₋₂₆₄ peptide in OT-1 mice, release T-cell and/or inflammatory effector cell derived mediator amounts which may be easily detectable by conventional assays.

We also have found that TCR transgenic mice, e.g. DO11.10 or OT-1 mice, may produce substantial amounts of IFN-γ upon stimulation by administration of (restricted) OVA-peptides with the respective TCR specificity. We further have found that in peptide stimulated DO11.10 mice endogenous production of IFN-γ before challenge with a triggering agent, such as an endotoxin, may even potentiate the production of pro-inflammatory cytokines such as e.g. IL-1α, IL-1β, and TNF-α.

In another aspect the present invention provides a kit (assay) of the present invention comprising as main components
a. a mouse wherein the majority of T cells express a transgenic MHC class I restricted or MHC class II restricted T cell receptor,
b. a MHC restricted TCR specific OVA-peptide,
c. optionally a triggering agent,
d. optionally controls, standards and/or calibration means,
e. optionally detection means for a T-cell and/or inflammatory effector cell derived mediator, and
f. instructions for using the components of said kit.

A kit (assay) of the present invention may further comprise a substantial component, e.g. including an appropriate environment of a sample to be tested.

Appropriate T-cell and/or inflammatory effector cell derived mediator-detection means include detection means according to a method as conventional, such as mediator detection with immunoassays such as e.g. all sorts of ELISAs (=enzyme linked immunosorbent assay), e.g. with an antibody which bears a label for detection or which bears no label. Such a label may be as conventional, e.g. an enzyme label, such as horse radish peroxidase (HRP) or peroxidase (POD); or a fluorescence label, e.g. a fluorescent dye. Such label may be detected as appropriate, e.g. according to a method as conventional, e.g. enzyme detection methods by carrying out an enzymatic reaction, or by fluorescence measurement. An antibody which bears no label may be detected as appropriate, e.g. indirectly, such as by use of a tandem system of a first antibody and a second antibody specifically recognizing the first antibody, which second antibody bears a label, e.g. an enzyme or fluorescence label, the label of which may be detected according to methods as conventional, e.g. as indicated herein.

A MHC restricted TCR specific OVA-peptide is preferably administered parenterally, e.g. intraperitoneal, intravenous or subcutaneous.

A triggering agent is understood to be an agent which stimulates the enhanced production of non-T-cell derived mediators, e.g. other inflammatory effector cell derived mediators, in a TCR transgenic mouse when stimulated before with the T specific peptide. An appropriate triggering agent includes a triggering agent as conventional, such as an endotoxin or a lipopolysaccharide of gram negative bacteria (LPS).

If a triggering agent is administered, the serum levels of pro-inflammatory and anti-inflammatory cytokines are determined, preferably the levels of IL-1α and/or IL-1β and/or IL-6 and/or TNF-α and/or IL-10.

In an OT-1 mouse with the specificity for a MHC class I restricted TCR specific peptide, the peptide is preferably OVA₂₅₇₋₂₆₄. The T cell mediator determined in an OT-1 mouse is preferably a CD8⁺ T cell derived cytokine, e.g. IL-2 and/or IFN-γ.

In a DO11.10 mouse with a specificity for a MHC class II restricted TCR specific peptide, the peptide is preferably OVA₃₂₃₋₃₃₉. The T cell mediator determined in a DO11.10 mouse is preferably a CD4⁺ T cell derived cytokine, e.g. one selected from the group consisting of IL-2, IL-4, IL-5, IL-13 and IFN-γ.

In another aspect the present invention provides a process for the determination of a T-cell and/or inflammatory effector cell derived mediator in serum of an OVA-peptide stimulated mouse wherein the majority of T cells express a transgenic MHC class I or MHC class II restricted T cell receptor, which process comprises the steps of
a. administering to a mouse wherein the majority of T cells express a transgenic MHC class I restricted or MHC class II restricted T cell receptor a MHC restricted TCR specific OVA-peptide and optionally a triggering agent, and
b. determining the level of a T-cell and/or inflammatory effector cell derived mediator produced.

If a DO11.10 mouse and a MHC restricted TCR specific OVA-peptide are used in a process of the present invention, preferably the mediator determined is a cytokine selected from the group consisting of IL-2, IL-4, IL-5, IL-13 and IFN-γ; and if additionally a triggering agent is administered, preferably the mediator determined is a cytokine selected from the group consisting of IL-1α, IL-1β, IL-6, IL-10 and TNF-α.

A kit or a process of the present invention may be useful in the provision of an agent that interferes with T cell activation and/or -differentiation and/or modulation of other inflammatory effector cells in vivo, which agent may be useful as a pharmaceutically active compound.

In another aspect the present invention provides a method for identifying, e.g. and using as a pharmaceutically active compound, an agent that interferes with T cell activation and/or -differentiation and/or modulation of other inflammatory effector cells comprising the steps of
a. administering to a mouse wherein the majority of T cells express a transgenic MHC class I or MHC class II restricted T cell receptor a MHC restricted TCR specific OVA-peptide and optionally a triggering agent,
b. administering to a transgenic mouse of step a) a candidate compound before, after or simultaneously with the peptide and optionally a triggering agent of step a),
c. determining the level of a T-cell and/or inflammatory effector cell derived mediator in serum of
   c1. a mouse treated according to step a), and
   c2. a mouse treated according to step b) and step a),
d. determining whether there is a difference in the level of said mediator produced in said serum, and
e. choosing an agent as determined in step d).

A candidate compound includes compound (libraries), from which the effect on T cell activation and/or -differentiation and/or modulation of other inflammatory effector cells is unknown. Compound (libraries) include for example oligopeptides, polypeptides, proteins, antibodies, (peptide-)mimetics, small molecules, e.g. low molecular weight compounds (LMW's). An agent is a candidate compound from which an effect on T cell activation and/or -differentiation and/or modulation of other inflammatory effector cells has been found in a screening assay or in a method for identifying an agent of the present invention. An agent may decrease or enhance the production of mediators from T cells or other inflammatory effector cells and includes agonists and antagonists in the production of such mediators. An agent includes compound(s)(libraries) from which its influence on T cells, e.g. CD8⁺ T cells and/or CD4⁺ T cells, and/or other inflammatory effector cells, e.g. monocytes/macrophages, via its influence on the release of appropriate mediators in vivo, can be determined. Compound (libraries) include for example oligopeptides, polypeptides, proteins, antibodies, (peptide-)mimetics, small molecules, e.g. low molecular weight compounds (LMW's). In case a candidate compound is administered, the candidate compound is preferably administered orally or parenterally.

In another aspect the present invention provides
a mouse wherein the majority of T cells express a transgenic MHC class I or MHC class II restricted T cell receptor for use in a method for the identification of an agent that interferes with T cell activation and/or -differentiation and/or modulation of other inflammatory effector cells;
the use of a mouse wherein the majority of T cells express a transgenic MHC class I or MHC class II restricted T cell receptor in a method for the identification of an agent that interferes with T cell activation and/or -differentiation and/or modulation of other inflammatory effector cells,
   e.g. comprising steps a. to e. as defined above.

In another aspect the present invention provides
the use of a mouse wherein the majority of T cells express a transgenic MHC class I or MHC class II restricted T cell receptor in a method for the determination of a T-cell and/or inflammatory effector cell derived mediator in the serum of said mouse;
   e.g. comprising the steps of a process for the determination of a T-cell and/or inflammatory effector cell derived mediator of the present invention.

In another aspect the present invention provides a kit of the present invention for use in a method for identifying an agent that interferes with T cell activation and/or -differentiation and/or modulation of other inflammatory effector cells.

Said kit may further comprise a substantial component, e.g. including an appropriate environment of a sample to be tested and, e.g. appropriate means to determine the effect of a candidate compound in a sample to be tested.

In another aspect the present invention provides an agent identified by a method comprising the steps of
a. administering to a mouse wherein the majority of T cells express a transgenic MHC class I or MHC class II restricted T cell receptor a MHC restricted TCR specific OVA-peptide and optionally a triggering agent,
b. administering to a transgenic mouse of step a) a candidate compound before, after or simultaneously with the peptide and optionally a triggering agent of step a),
c. determining the level of a T-cell and/or inflammatory effector cell derived mediator in serum of
   c1. a mouse treated according to step a) and
   c2. a mouse treated according to step b) and step a),
d. determining whether there is a difference in the level of said mediator produced in said serum, and
e. choosing an agent as determined in step d).

An agent of the present invention may exhibit pharmacological activity and is therefore useful as a pharmaceutical, e.g. in the treatment of diseases which are mediated by T-cell and/or inflammatory effector cell derived mediators. Inhibitory agents might be useful as a pharmaceutical for e.g. diseases based on an unwanted or aberrant immune response, such as allergic diseases, transplantation, autoimmune related and inflammatory diseases. Allergic disease include e.g. allergic asthma, contact allergy, drug allergy, food allergy, atopic dermatitis or seasonal allergies such as allergic rhinitis. Transplantation includes transplantation of solid organs, skin, cornea transplantation or bone marrow transplantation. Autoimmune related and inflammatory diseases include e.g. type I diabetes, multiple sclerosis, rheumatoid arthritis, psoriasis, inflammatory bowel disease, atherosclerosis or systemic Lupus erythematosus.

Stimulatory agents might be useful in all situations where the immune response should be strengthened, e.g. infections, immunodeficiencies or tumors.

In another aspect the present invention provides
an agent of the present invention for use as a pharmaceutical, e.g. for the treatment of a disease which is based on an unwanted or aberrant immune response selected from the group consisting of allergic disease, transplantation, autoimmune related disease, inflammatory disease and modulation/stimulation of a tumor specific or pathogen specific response, and
a pharmaceutical composition comprising at least one agent that interferes with T cell activation and/or -differentiation and/or modulation of other inflammatory effector cell identified by a method according to the present invention beside pharmaceutically acceptable excipient(s).

Pharmaceutically acceptable excipient(s) include e.g. appropriate carrier and/or diluent, e.g. including fillers, binders, disintegrators, flow conditioners, lubricants, sugars and sweeteners, fragrances, preservatives, stabilizers, wetting agents and/or emulsifiers, solubilizers, salts for regulating osmotic pressure and/or buffers.

In another aspect the present invention provides a method for the treatment of a disease which is based on an unwanted or aberrant immune response selected from the group consisting of allergic disease, transplantation, autoimmune related disease, inflammatory disease and modulation/stimulation of a tumor specific or pathogen specific response, comprising administering an agent identified by a method of the present invention or a pharmaceutical composition of the present invention to a subject in need of such a treatment.

In another aspect the present invention provides a method for the treatment of a disease which is based on an unwanted or aberrant immune response selected from the group consisting of allergic disease, transplantation, autoimmune related disease, inflammatory disease and modulation/stimulation of a tumor specific or pathogen specific response, wherein the
allergic disease is allergic asthma, contact allergy, drug allergy, food allergy, atopic dermatitis or seasonal allergy,
transplantation is transplantation of solid organs, skin, cornea or bone marrow,
the autoimmune related and inflammatory disease is type I diabetes, multiple sclerosis, rheumatoid arthritis, psoriasis, inflammatory bowel disease, atherosclerosis or systemic Lupus erythematosus,
the tumor specific or pathogen specific response is infection, immunodeficiencies or tumor treatment.

An agent of the present invention for treatment includes one or more, preferably one, agent of the present invention, e.g. a combination of two or more agents of the present invention. Treatment includes treatment and prophylaxis.

In another aspect the present invention provides the use of an agent of the present invention for the manufacture of a medicament, e.g. a pharmaceutical composition, for the treatment of diseases which are mediated by T-cell and/or inflammatory effector cell derived mediators, e.g. diseases based on an unwanted or aberrant immune response, such as allergic diseases, transplantation, autoimmune related and inflammatory diseases or in situations where the immune response should be strengthened, e.g. infections, immunodeficiencies, tumors and the use as vaccines (adjuvants).

For such treatment (use), the appropriate dosage will, of course, vary depending upon, for example, the chemical nature and the pharmacokinetic data of an agent of the present invention employed, the individual host, the mode of administration and the nature and severity of the conditions being treated. However, in general, for satisfactory results in larger mammals, for example humans, an indicated daily dosage is in the range from about 0.01 g to about 2.0 g (e.g. about 0.125 mg/kg to about 25 mg/kg), such as 0.05 to 2.0 g, e.g. 0.1 to 0.5 mg, of an agent of the present invention; conveniently administered, for example, in divided doses up to four times a day.

An agent of the present invention may be administered by any conventional route, for example enterally, e.g. including nasal, buccal, rectal, oral administration; parenterally, e.g. including intravenous, intramuscular, subcutanous administration; or topically; e.g. including epicutaneous, intranasal, intratracheal administration;

e.g. in form of coated or uncoated tablets, capsules, injectable solutions or suspensions, e.g. in the form of ampoules, vials, in the form of creams, gels, pastes, inhaler powder, foams, tinctures, lip sticks, drops, sprays, or in the form of suppositories.

An agent of the present invention may be administered in the form of a pharmaceutically acceptable salt, e.g. an acid addition salt or metal salt; or in free form; optionally in the form of a solvate. An agent of the present invention in the form of a salt may exhibit the same order of activity as an agent of the present invention in free form; optionally in the form of a solvate.

An agent of the present invention may be used for pharmaceutical treatment according to the present invention alone, or in combination with one or more other pharmaceutically active agents.

Combinations include fixed combinations, in which two or more pharmaceutically active agents are in the same formulation; kits, in which two or more pharmaceutically active agents in separate formulations are sold in the same package, e.g. with instruction for co-administration; and free combinations in which the pharmaceutically active agents are packaged separately, but instruction for simultaneous or sequential administration are given.

In another aspect the present invention provides a pharmaceutical composition according to the present invention, further comprising another pharmaceutically active agent.

Such compositions may be manufactured according, e.g. analogously to a method as conventional, e.g. by mixing, granulating, coating, dissolving or lyophilizing processes. Unit dosage forms may contain, for example, from about 0.5 mg to about 2000 mg, such as 1 mg to about 500 mg, e.g. 0.00625 mg/kg to about 12.5 mg/kg.

In another aspect the present invention provides a method for identifying, e.g. and using as a pharmaceutical, an agent that interferes with T-cell activation and/or differentiation, esp. with CD8⁺ T-cells, comprising the steps of
a) providing a mouse wherein the majority of T-cells express a transgenic MHC class I restricted T-cell receptor, e.g. an OT-1 mouse,
b) administering to a transgenic T-cell receptor carrying mouse according to step a) the respective T-cell receptor specific peptide, e.g. OVA₂₅₇₋₂₆₄ peptide in an OT-1 mouse, in a defined amount,
c) administering to a transgenic mouse of step b) a candidate compound which might interfere with T-cell activation/differentiation, esp. with CD8⁺ T-cells, before, after or simultaneous with the peptide of step b), in a defined amount,
d) determining the level of a T-cell derived cytokine, e.g. a CD8⁺ T-cell derived cytokine, preferably IL-2 and IFN-γ, in serum of candidate compound treated mouse according to steps a)-c) versus candidate compound untreated mouse according to steps a) and b) and determining whether there is a difference in the cytokine level, and
e) choosing an agent from said candidate compound as determined in step d), e.g. for use as a pharmaceutical.

In another aspect the present invention provides a method for identifying, e.g. and using as a pharmaceutical, an agent that interferes with T-cell activation and/or differentiation, esp. with CD4⁺ T-cells, comprising the steps of
a) providing a mouse wherein the majority of T-cells express a transgenic MHC class II restricted specific T-cell receptor, e.g. a DO11.10 mouse,
b) administering to a transgenic T-cell receptor carrying mouse according to step a) the respective T-cell receptor specific peptide, e.g. OVA₃₂₃₋₃₃₉ peptide in a DO11.10 mouse, in a defined amount,
c) administering to a transgenic mouse according to step b) a candidate compound which might interfere with T-cell activation and/or differentiation, esp. with CD4⁺ T-cells, before, after or simultaneous with the peptide of step b), in a defined amount,
d) determining the level of a T-cell derived cytokine, e.g. a CD4⁺ T-cell derived cytokine, preferably IL-2, IL-4 and/or INF-γ, in serum of candidate compound treated mouse according to steps a)-c) versus candidate compound untreated mouse according to steps a) and b) and determining whether there is a difference in the cytokine level, and
e) choosing an agent from said candidate compound as determined in step d), e.g. for use as a pharmaceutical.

In another aspect the present invention provides a method for identifying, e.g. and using as a pharmaceutical, an agent that interferes with T cell derived cytokine mediated cell activation, comprising the steps of
a) providing a mouse wherein the majority of T-cells express a transgenic MHC class II or MHC class I restricted T-cell receptor, e.g. a DO11.10 or an OT-1 mouse, b) administering to a transgenic mouse according to step a) a T-cell receptor specific peptide as a priming agent, e.g. OVA₃₂₃₋₃₃₉ peptide in a DO11.10 mouse or OVA₂₅₇₋₂₆₄ peptide in an OT-1 mouse, in a defined amount,
c) administering to a transgenic mouse according to steps a) and b) a candidate compound which might interfere with T cell derived cytokine mediated cell activation before, after or simultaneous with the priming agent of step b) in a defined amount,
d) administering to a transgenic mouse of steps a) to c) as a triggering agent an endotoxin/lipopolysaccharide from gram negative bacteria in a defined amount,
e) determining the level of an inflammatory cytokine and/or chemokine and/or another mediator, preferably IL-1α, IL-1β, IL-6, TNF-α and/or IL-10, in serum of candidate compound treated mouse according to steps a)-d) versus candidate compound untreated mouse according to steps a), b) and d) and determining whether there is a difference in a cytokine and/or chemokine and/or another mediator level,
   f) choosing an agent from said candidate compound as determined in step e), e.g. for use as a pharmaceutical.

### DESCRIPTION OF THE FIGURES

FIG. 1: Groups of sex-matched OT-1 mice are given 100 μg/mouse of OVA₂₅₇₋₂₆₄ in 0.2 ml PBS i.p. At indicated time points, peripheral blood and splenocytes are harvested and stained for the expression of activation markers and on CD8⁺ T cells. Data is shown as values of individual mice.

FIG. 2: Groups of sex-matched OT-1 mice are given indicated amounts of OVA₂₅₇₋₂₆₄ in 0.2 ml PBS i.p. 4 hours later, peripheral blood is collected and serum is analyzed for IFN-γ and IL-2. Data is expressed as values of individual mice.

FIG. 3: Groups of sex-matched OT-1 mice are given CsA p.o. in Neoral placebo at the indicated dose. 2 hours after compound administration, animals are immunized with 30 μg/mouse of OVA₂₅₇₋₂₆₄ in 0.2 ml PBS i.p. The negative control group (PBS) receive 0.2 ml PBS i.p. only. After 4 hours blood stained for the expression of activation markers CD25 (left) and CD69 (right) on CD8⁺ T cells. Data is expressed as individual values per mouse with a horizontal bar giving the mean value per group. Stars indicate significant differences (p<0.05) to the vehicle-treated, peptide-primed control group (Peptide). § denotes a mouse that was poorly primed.

FIG. 4: shows the corresponding effect on the serum levels of IFN-γ and IL-2 after CsA treatment. Interestingly, a low dose of CsA (0.1 mg/kg b.w.) leads to an increase in both IFN-γ as well as IL-2 levels, whereas an increased amount of CsA (10 mg/kg b.w.) leads to the complete abrogation of the cytokines in the serum.

In the following examples temperatures are given in degree Celcius (° C.) and are uncorrected.

The following ABBREVIATIONS are used:
b.w. body weight
FACS fluorescence-activated cell sorting
IFN-γ interferon-γ
IL interleukin
i.p. intraperitoneal
mAb monoclonal antibody
CsA cyclosporin A
LPS lipopolysaccharide
OVA chicken ovalbumin
OVA₂₅₇₋₂₆₄ peptide derived from chicken ovalbumin containing amino acids from position 257 to 264
OVA₃₂₃₋₃₃₉ peptide derived from chicken ovalbumin containing amino acids from position 323 to 339
PE phycoeythrin
FITC fluorescein isothiocyanate
TCR T cell receptor
TMB tetramethylbenzidine
TNF-α tumor necrosis factor-α

### EXAMPLES

### Example 1

### OT-1 Mice

A) Experimental Setup

a) Laboratory Animals

OT-1 mice are obtained as described in Hogquist et al., 1994, Cell 76(1)17-27. The animals are maintained in Microvent positive air supply cages (22±1° C., 55±5% relative humidity, 50-70 changes of fresh air/hour and 12 hours day and night cycle) with sawdust bedding. Standard laboratory mouse chow (SNIFF, Soest, Germany) and drinking water is given ad libitum. Groups of two to four, 8-10 weeks old, male or female OT-1 mice weighing between 20-25 g are used for the experiments described. The animals are grouped (4 per group) in type II Macrolon cages one day before the experiment.

b) T Cell Proliferation Assay

Splenocytes (4×10⁵ cells/well) of OT-1 mice are stimulated in 200 μl final volume in 96-well flat bottomed plates with titrated amounts of OVA or OVA₂₅₇₋₂₆₄ peptide at 37°. As medium RPMI 1640 supplemented with 10% FCS, 2.0 g/l NaHCO₃, 2 mM L-glutamin, 100 IU/ml penicillin, 100 μg/ml streptomycin and 5×10⁻⁵ M β-mercaptoethanol (Sigma, Vienna, Austria) is used. After 48 hours, cells are pulsed with 0.5 μCi ³H-thymidine for 8 hours and the incorporated radioactivity is measured on a liquid scintillation β-counter (Trilux, 1450 Microbeta, Wallac, Turku, Finland).

c) Cytokine Production Assay

Cells are stimulated as described above and supernatant is harvested after 48 hours. The quantity of cytokines released by T cells into the culture medium after antigen stimulation is assayed by conventional sandwich ELISA. Microtiter plates (Nunc-Immunoplate™) are coated with the cytokine-specific capture antibodies JES6-1A12 (anti-IL-2), 11B11 (anti-IL-4), TRFK5 (anti-IL-5), JES5-2A5 (anti-IL-10) or R4-6A2 (anti-IFN-γ) at 4° overnight. Supernatants are incubated on the coated plates for 2 hours. Specifically bound cytokines are quantified using biotinylated secondary antibodies JES6-5H4 (anti-IL-2), BVD6-24G2 (anti-IL-4), TRFK4 (anti-IL-5), SXC-1 (anti-IL-10) and XMG1.2 (anti-IFN-γ) followed by extravidin peroxidase and TMB substrate (both from Sigma). Absorption is measured at 450 and 690 nm on a Spectramax 340PC reader (Molecular Devices, Sunnyvale, Calif.). All cytokines are quantified with standard curves obtained with known amounts of recombinant mouse cytokines using SoftMax 3 software. All mAbs and recombinant mouse cytokine standards are purchased from BD PharMingen, Heidelberg, Germany.

d) Treatment and Cell Preparation

CsA is used as Neoral® (commercial drinking solution 100 mg/ml Novartis Pharma GmbH, Vienna). Doses of 3, 0.3 and 0.1 mg/kg b.w. are administered orally by gavage 2 hours before antigen challenge. The compound is diluted with solvent (Neoral Placebo) and given in a volume of 0.2 ml. Animals in the control group receive the solvent only. Ovalbumin-derived peptide (OVA₂₅₇₋₂₆₄, SIINFEKL, >80% purity, piChemie R&D, Graz, Austria) or whole OVA (Sigma) are used as antigens. Both are diluted in PBS and administerd i.p. in a final volume of 0.2 ml. At various time points after OVA₂₅₇₋₂₆₄ peptide injection about 200 μl blood is withdrawn from the retro-orbital plexus under ether anesthesia. In order to avoid coagulation, blood is collected in EDTA-containing buffer. Mice are killed by cervical dislocation and the spleen is excised. Single cell suspension are prepared in PBS by gently pressing the spleen through a 100 μm cell strainer. Finally, blood and spleen cells are washed with FACS buffer (PBS, 1 g/l EDTA, 1 g/l NaN₃, 10% FCS; pH 7.1) and taken up in the same buffer for FACS analysis.

e) FACS Analysis

Single cell suspensions of spleen or blood containing approximately 1×10⁶ cells in 100 μl of FACS buffer are used for staining. Non-specific binding is blocked by addition of 2 μl of unlabeled Fcγ III/II receptor-blocking Ab (anti-CD16/CD32, clone CT-17.1, 17.2). For phenotypic analysis, samples are stained with PE-labeled anti-CD8 mAb (clone CT-CD8a) in conjunction with FITC-labeled anti-CD4 mAb (clone CT-CD4), PE-labeled anti-CD8 mAb in conjunction with FITC-labeled anti-TCR Vβ5.1,5.2 antibody (clone MR9-4) or FITC-labeled anti-CD8 mAb in conjunction with PE-labeled anti-TCR Vβ2 antibody (clone B20.1). For detection of activation markers cells are stained with FITC-labeled anti-CD8 mAb (clone CT-CD8a) in conjunction with either PE-labeled anti-CD25 mAb (clone PC61 5.3) or PE-labeled anti-CD25 mAb (clone H1.2F3). All antibodies except for anti-TCR Vβ5 and Vα2 are obtained from Caltag, Burlingame, Calif. Those are purchased from BD PharMingen. Blood cells are treated after antibody staining with NH₄Cl buffer (PharM Lyse, BD PharMingen). Cells are washed in FACS buffer and data is acquired on a FACS Calibur (Becton Dickinson) flow cytometer using Celiquest Plus software. Lymphocytes are gated in the FSC/SSC dot blot according to size and granularity and further analyzed for expression of CD8, TCR Vβ5 expression and activation markers. Data are presented as original dot blots or are calculated from dot blots as percentage of cells positively stained for TCR or activation markers within the CD8⁺ population.

Data calculated from FACS analysis or serum cytokine determination are given as individual values for each animal. Data are analysed for normal distribution and then the ANOVA test for multiple comparison is applied using SigmaStat software. Statistical significance is taken as p<0.05.

B) Results

a) Kinetics of Peptide-Induced T Cell Activation In Vivo

Upon i.p. treatment of mice with 100 μg of the immunogenic peptide OVA₂₅₇₋₂₆₄ the frequency of CD8⁺ expressing cells among the lymphocyte population in the blood drops from around 30% to less than 2.5% within 3 hours. CD8⁺ T cell numbers remain at this low level for 24 hours and then recover to nearly pre-experiment levels after 48 hours. In the spleen no significant decrease of CD8⁺ T cells is observed.

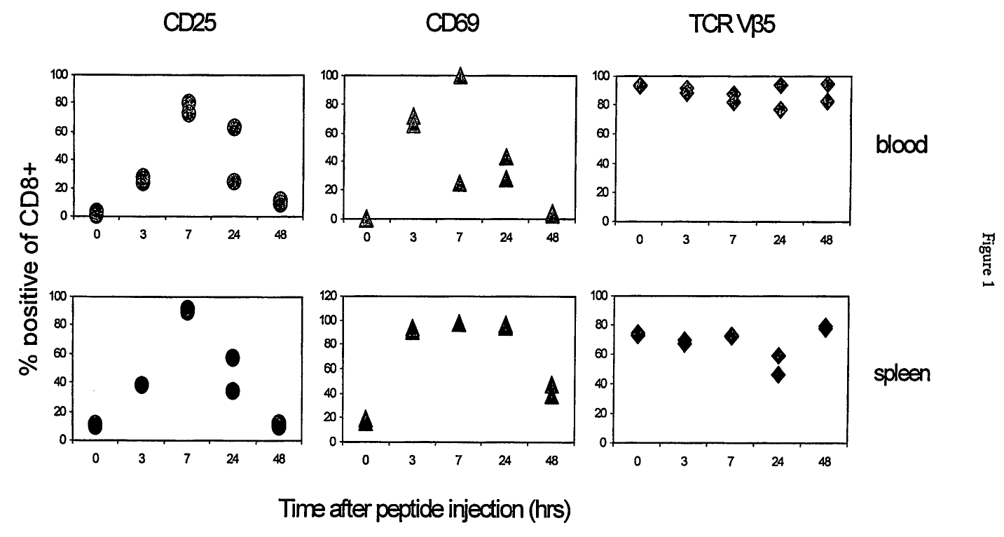

Administration of 100 μg of OVA₂₅₇₋₂₆₄ induces the up-regulation of CD25 and CD69 on both spleenic CD8⁺ T cells and the low number of remaining blood CD8⁺ T cells. CD25 is detectable after 3 hours, maximal (ca. 40-fold increase) after 7 hours and reaches background levels after 48 hours. CD69 induction is more rapid with maximal levels (ca. 155-fold increase) reached already after 3 hours and is sustained for more than 24 hours. Antigen-mediated activation by peptide injection does not lead to a significant down regulation of the transgenic TCR (see FIG. 1).

Peptide-induced activation in OT-1 mice is accompanied by a transient burst of the inflammatory cytokines IFN-γ and IL-2 in the serum. Both cytokines show an early peak at 4 hours, weaning off by 8 hours and reach background levels after 24 hours.

Although high amounts of OVA protein induce proliferation and cytokine release of OT-1 splenocytes in vitro, i.p. administration of 1 mg of OVA results only in a late (7-24 hours) and low increase (max. 2.5-fold) of CD69 expression in the spleen but not the blood. The frequency of CD8⁺ T cells in both spleen and blood declines marginally over 48 hours.

b) Dependence of Activation on Peptide Amounts

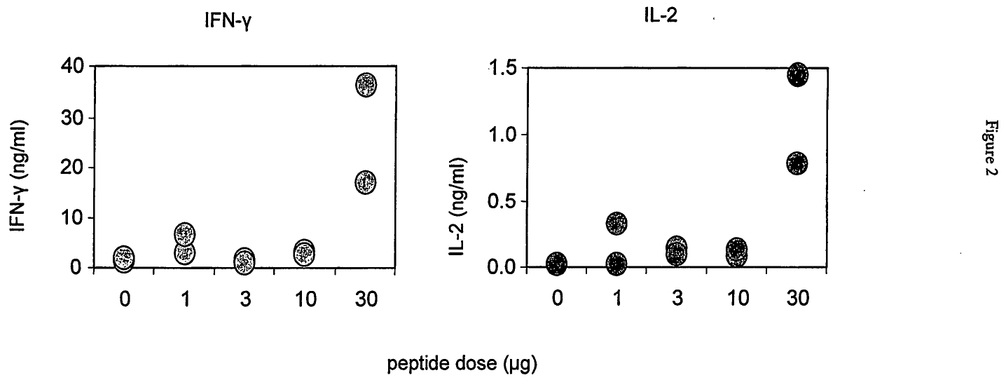

The influence of antigen amount on CD8⁺ T cell activation is determined in order to estimate the threshold concentration for activation in vivo and to establish an optimal amount for a routine model (see FIG. 2). A minimal amount of 30 μg of OVA₂₅₇₋₂₆₄ per animal is required to induce significant serum quantities of both IFN-γ and IL-2.

The extent of T cell activation also correlates with the administered peptide amount with a maximal response at 30 μg of OVA₂₅₇₋₂₆₄ per animal.

c) Effect of CsA on Peptide-Induced T Cell Activation

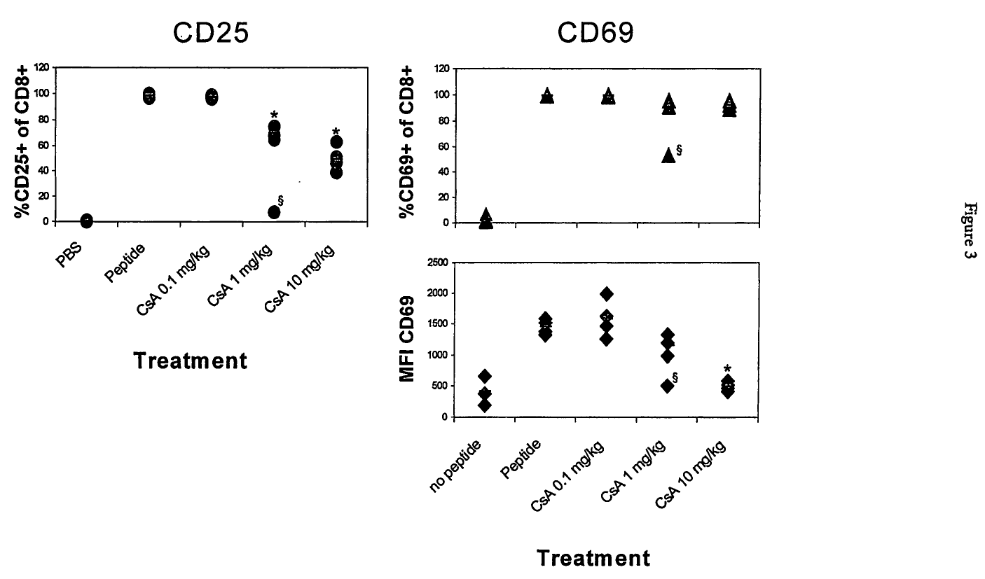

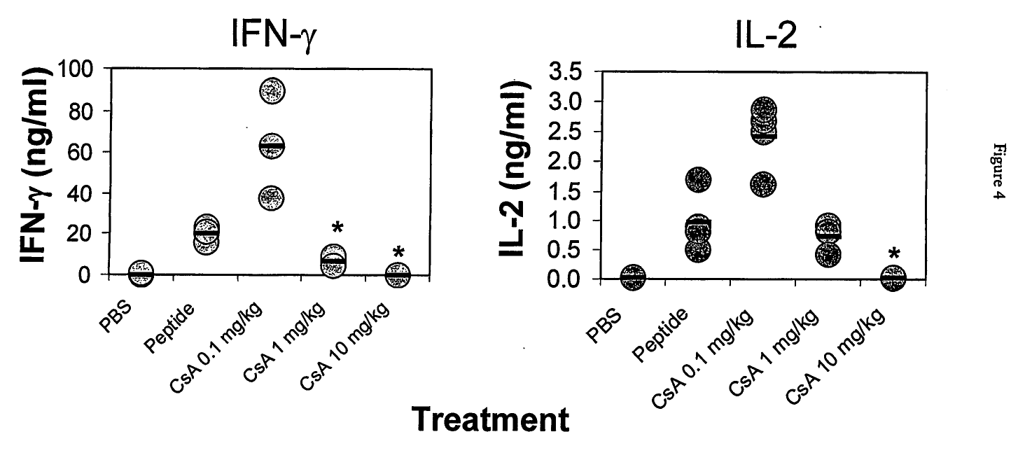

In order to validate the model, various amounts of immunosuppressant CsA are used to interfere with T cell activation. The spleen is chosen as the target organ for analysis of activation and the peptide is given in an amount of 30 μg/mouse. Because maximal activation is already seen after 4 hours post peptide administration, this time point is selected for analysis. As shown in FIG. 3, CsA significantly inhibits peptide-induced CD25 expression at a dose of 1 mg/kg b.w. (30.1%±5.2% inhibition) and 10 mg/kg b.w. (49.5%±10% inhibition). After 4 hours blood is taken and serum analyzed for IFN-γ and IL-2 in a sandwich ELISA (see FIG. 4).

### Example 2

### DO11.10 Mice

A) Experimental Setup

a) Laboratory Animals

Groups of six, 8-10 weeks old, male or female DO11.10 mice weighing between 20-22 g are obtained as described in Murphy K. M. et al. 1990, Science 250:1720-1722. The animals are maintained in Microvent positive air supply cages (22±1° C., 55±5% relative humidity, 50-70 changes of fresh air/hour and 12 hours day and night cycle) with sawdust bedding. Standard laboratory mouse chow (SNIFF, Soest, Germany) and drinking water is given ad libidum. The animals are used for the study after an adaptation period of 10-14 days and grouped (6 per group) in type II Macrolon cages one day before the experiment.

b) Administration of OVA or OVA₃₂₃₋₃₃₉ Peptide

Groups of six DO11.10 mice are i.p. injected with 10 μg of ovalbumin (OVA, Sigma, St. Louis, USA) absorbed to 2 mg of Al(OH)₃ (Alu Gel S; 2% Al(OH)₃, Serva, Heidelberg, FRG) and diluted with saline to a volume of 0.2 ml or various amounts (1, 3, 10, 30, or 90 μg per mouse) of alum-precipitated OVA₃₂₃₋₃₃₉ peptide (98% purity, piChem R&D, Graz Austria) in 0.2 ml on days 0 and 14. Before and at different time points on days 0 and 14 after the OVA or OVA₃₂₃₋₃₃₉ peptide administration about 200 μl of blood are collected by puncture of the retro-orbital plexus under ether anesthesia.

The sera are collected and stored frozen at −20° C. until analyzed.

c) Treatment with CsA

CsA (Neoral 100 mg/ml Novartis Pharma GmbH, Vienna) is administered orally by gavage. The Neoral solution is diluted further with tap water. Doses of 20, 40, and 80 mg/kg b.w. are administered in a volume of 0.2 ml. Animals in the control group receive the solvent alone.

d) Quantification of Cytokines in Mouse Sera

Murine IL-2, IFN-γ and IL-4 are analyzed by immunoassays (Quantikine R&D Systems, Minneapolis, Minn., USA). Results are expressed as pg per ml serum expressed as mean±SEM pg of the cytokine indicated per ml serum. Test and control groups are evaluated by Dunn test by Kruskall-Wallis One Way Anova using the SigmaStat® program.

B) Results

a) Sensitization with OVA₃₂₃₋₃₃₉ Peptide Versus OVA

Serum INF-γ levels in DO11.10 mice after sensitization with OVA protein (=OVA) or OVA₃₂₃₋₃₃₉ peptide are shown in TABLE 1. On days 0 and 14 groups of 6 DO11.10 mice are injected i.p. either with alum/alum (a/a), 10 μg OVA/10 μg OVA (o/o), 10 μg OVA/10 g OVA₃₂₃₋₃₃₉ peptide (o/p) or 10 μg OVA₃₂₃₋₃₃₉ peptide/10 μg OVA₃₂₃₋₃₃₉ peptide (p/p). Blood is collected at 3 hours after antigen injection on days 0 and 14. INF-γ levels are analyzed in the sera by an ELISA. Results are expressed as mean pg per ml serum±SEM.

TABLE 1

| | |
|---|---|
| administration | IFN-γ level in pg/ml serum after |

| scheme | day 0 | day 14 |
|---|---|---|
| a/a | 12.077 +/− 7.455 | 32.203 +/− 2.201 |
| o/o | 3.788 +/− 1.495 | 6.796 +/− 6.189 |
| o/p | 14.023 +/− 3.871 | 577.546 +/− 148.064 |
| p/p | 483.215 +/− 134.641 | 554.136 +/− 110.652 |

The i.p. administration of alum alone results in an about 4 fold increase in IFN-γ levels within 3 hours after the injection. A second injection of alum 2 weeks later induces a similar rise in IFN-γ levels in the sera. The primary sensitization with 10 μg of OVA does not change the serum INF-γ levels above those seen with alum alone. However, when these mice receive 10 μg of OVA₃₂₃₋₃₃₉ peptide 2 weeks later, about a 25 fold rise in the INF-γ serum levels can be measured compared to mice which receive alum alone. A primary sensitization with OVA followed 2 weeks later by a second OVA exposure fail to increase the INF-γ levels about those determined in the sera before the second sensitization. Mice injected for the first time with 10 μg of OVA₃₃₋₃₃₉ peptide respond with a 50 fold increase in IFN-γ levels in their sera 3 hours after the peptide injection. The second sensitization with the same dose of OVA₃₂₃₋₃₃₉ peptide induces a rise of the INF-γ levels of the same magnitude.

These results show that an increase in serum INF-γ levels can be induced in DO11.10 mice by sensitization with the OVA₃₂₃₋₃₃₉ peptide but the serum levels are under the detection limit when mice are challenged with the given amount of OVA-protein.

b) Serum Cytokine Levels Upon Stimulation with Various Doses of OVA₃₂₃₋₃₃₉ Peptide

1) Changes in Serum INF-γ Levels

Serum INF-γ levels after primary and secondary stimulation with various doses of the OVA₃₂₃₋₃₃₉ peptide are summarized in TABLE 2: Groups of 6 mice receive various doses (1, 3, 10, 30 and 90 μg/mouse) of alum-precipitated peptide i.p. on day 0 and 14. Mice of the control group are injected with alum. Blood is collected before and at 2, 4, and 6 hours after the first peptide injection. INF-γ is determined in the sera by an ELISA. Results are expressed as mean pg per ml serum±SEM.

TABLE 2

| administration | IFN-γ level in pg/ml serum on day 0 after different time points | | | |
|---|---|---|---|---|
| scheme | after 0 hours | after 2 hours | after 4 hours | after 6 hours |
| alum | 3.559 +/− 1.296 | 7.256 +/− 3.133 | 10.347 +/− 1.423 | 14.166 +/− 2.464 |
| 1 μg OVA₃₂₃₋₃₃₉ | 11.181 +/− 1.288 | 18.047 +/− 3.944 | 41.034 +/− 12.394 | 15.925 +/− 4.594 |
| 3 μg OVA₃₂₃₋₃₃₉ | 8.230 +/− 1.430 | 62.724 +/− 7.939 | 63.023 +/− 12.462 | 25.962 +/− 8.858 |
| 10 μg OVA₃₂₃₋₃₃₉ | 0.374 +/− 0.324 | 65.927 +/− 23.147 | 202.260 +/− 48.410 | 24.047 +/− 9.455 |
| 30 μg OVA₃₂₃₋₃₃₉ | 3.988 +/− 0.700 | 207.727 +/− 62.093 | 176.807 +/− 79.268 | 187.325 +/− 62.370 |
| 90 μg OVA₃₂₃₋₃₃₉ | — | 407.899 +/− 106.513 | 413.925 +/− 65.247 | 514.918 +/− 72.758 |

In the control group, receiving alum alone, INF-γ levels do not change significantly when blood is collected before and at 2, 4, and 6 hours after the injection. The primary stimulation with a dose of 1 μg of OVA₃₂₃₋₃₃₉ peptide per mouse induces a small increase in serum INF-γ levels only at 4 hours. A dose of 3 μg of OVA₃₂₃₋₃₃₉ peptide increases serum INF-γ levels about 11 fold at 2 and 4 hours compared to the control group receiving alum diluted with saline. IFN-γ levels in the serum increase about 40 fold at 4 hours after injecting 10 g of OVA₃₂₃₋₃₃₉ peptide while a 30 μg of OVA₃₂₃₋₃₃₉ peptide dose per mouse results in a 32-38 fold increase at 2, 4 and 6 hours after peptide injection. Increasing the dose to 90 μg of peptide per mouse increases the INF-γ serum concentration 75 fold at 2 and 4 hours and 94 fold at 6 hours after injection. A similar picture of the rises of the serum IFN-γ levels is seen when the animals are stimulated with the same doses of the OVA₃₂₃₋₃₃₉ peptide 2 weeks later.

2) Serum IL-4 Levels

Serum IL-4 levels after primary and secondary OVA₃₂₃₋₃₃₉ peptide antigen stimulation are shown in TABLE 3: Groups of 6 mice receive various doses (1, 3, 10, 30 and 90 μg/mouse) of alum-precipitated OVA₃₂₃₋₃₃₉ peptide i.p. on day 0 and 14. Animals of the control group are injected with alum. Blood is collected before and at 2, 4, and 6 hours after the first (A) and after the second peptide injection (B). IL-4 is determined in the sera by an ELISA. Results are expressed as mean pg per ml serum±SEM.

TABLE 3

| administration | IL-4 level in pg/ml serum on day 0 after | | | |
|---|---|---|---|---|
| scheme | 0 hours | 2 hours | 4 hours | 6 hours |
| alum | 14.865 +/− 1.384 | 31.994 +/− 5.465 | 19.173 +/− 1.902 | 17.891 +/− 1.914 |
| 1 μg OVA₃₂₃₋₃₃₉ | 25.770 +/− 2.618 | 36.426 +/− 5.871 | 4.094 +/− 1.072 | 13.354 +/− 4.010 |
| 3 μg OVA₃₂₃₋₃₃₉ | 17.905 +/− 3.616 | 37.706 +/− 4.028 | 7.903 +/− 0.957 | 11.051 +/− 2.549 |
| 10 μg OVA₃₂₃₋₃₃₉ | 16.296 +/− 3.397 | 41.870 +/− 9.751 | 18.804 +/− 1.757 | 5.735 +/− 2.226 |
| 30 μg OVA₃₂₃₋₃₃₉ | 16.524 +/− 2.209 | 93.594 +/− 29.015 | 41.725 +/− 10.226 | 17.173 +/− 2.069 |
| 90 μg OVA₃₂₃₋₃₃₉ | — | 273.017 +/− 95.997 | 146.940 +/− 21.784 | 127.616 +/− 7.397 |

In mice of the control group, injected with alum, IL-4 levels remain unchanged as is also seen in the mice which receive 1 μg of peptide. Doses of 3 μg and 10 μg of peptide increase serum IL-4 levels only slightly after 2 hours compared to the control animals injected with alum. Serum IL-4 levels rise 3 fold at 2 hours after the administration of a dose of 30 μg of peptide while a 90 μg peptide dose induces a 14 fold increase after 2 hours and a 7-8 fold rise after 4 and 6 hours. The second injection of 1 μg of peptide 2 weeks later does not change the IL-4 levels significantly when compared to the control.

3) Serum IL-2 Levels

Serum IL-2 levels after primary OVA₃₂₃-339 peptide antigen stimulation are shown in TABLE 4: Groups of 6 mice receive various doses (3, 10, 30 and 90 μg/mouse) of alum-precipitated OVA₃₂₃₋₃₃₉ peptide i.p. on day 0. Animals of the control group are injected with alum. Blood is collected before and at 2 hours after the peptide injection. IL-2 is determined in the sera by an ELISA. Results are expressed as mean pg per ml serum±SEM.

TABLE 4

| | IL-2 levels in pg/ml serum on day 0 after |
|---|---|
| administration of | 2 hours |
| alum alone | 135.939 +/− 17.348 |
| 3 μg OVA₃₂₃₋₃₃₉ | 327.513 +/− 76.331 |
| 10 μg OVA₃₂₃₋₃₃₉ | 2866.966 +/− 361.613 |
| 30 μg OVA₃₂₃₋₃₃₉ | 2926.303 +/− 381.536 |
| 90 μg OVA₃₂₃₋₃₃₉ | 4841.034 +/− 108.352 |

The results of this study demonstrate a dose/amount-dependent rise in serum IFN-γ, IL-4 and IL-2 levels with maximum values at several hours after the injection of the OVA₃₂₃₋₃₃₉ peptide. It seems that even with a dose of 90 μg of peptide, the plateau of the dose-response relationship of the serum cytokine levels is not reached. A dramatic rise in the IL-4 levels up to several 100 fold can be seen after the second antigen stimulation.

c) Sensitization with CsA Before Stimulation with OVA₃₂₃₋₃₃₉ Peptide

The influence of CsA on cytokine production in DO11.10 mice after stimulation with the OVA₃₂₃₋₃₃₉ peptide is given in TABLE 5: Groups of 6 DO11.10 mice are pretreated orally with various doses (10, 20, 30, 40, 80 mg/kg) of CsA. One hour later 30 μg of alum precipitated OVA₃₂₃₋₃₃₉ peptide is injected i.p. Animals of the control group receive the solvent (placebo) of CsA. Blood is collected 2 hours later and IFN-γ, IL-4 and IL-2 levels are determined in the sera by ELISAs. Results are expressed as mean pg per ml serum±SEM.

TABLE 5

| administration | level of IFN-γ in pg/ml | level of IL-4 | level of IL-2 |
|---|---|---|---|
| of | serum | in pg/ml serum | in pg/ml serum |
| placebo | 468.721 +/− 79.315 | 342.129 +/− 71.157 | 3219.232 +/− 231.559 |
| 10 mg/kg CsA | 423.781 +/− 118.481 | 111.515 +/− 37.08 | 2842.870 +/− 357.515 |
| 20 mg/kg CsA | 56.675 +/− 19.957 | 3.982 +/− 3.093 | 758.934 +/− 149.960 |
| 30 mg/kg CsA | 35.396 +/− 11.366 | 36.789 +/− 9.829 | 297.635 +/− 24.920 |
| 40 mg/kg CsA | 8.958 +/− 5.351 | 5.339 +/− 2.552 | 149.981 +/− 30.115 |
| 80 mg/kg CsA | 17.376 +/− 15.214 | 22.882 +/− 8.417 | 121.848 +/− 38.565 |

As can be seen in TABLE 5, the IL-2 levels in the sera of mice receiving the CsA solvent increases from pretreatment values below the detection limit to a mean value of 3219±231 pg/ml serum within 2 hours after the administration of 30 μg of OVA₃₂₃₋₃₃₉ peptide. Pretreatment with an oral dose of 10 mg of CsA/kg 1 hour before OVA₃₂₃₋₃₃₉ peptide reduces the IL-2 content by only 12% while 20 mg/kg reduces the IL-2 concentration by 76%. The IL-2 content of the sera when the mice are treated with 30 mg of CsA/kg b.w. is only 9% of the control group. Doses of 40 and 80 mg/kg reduce the IL-2 content in the sera to about 5%. The reduction in the IFN-γ values in response to the CsA treatment is also shown. Within 2 hours after the administration IFN-γ serum level in the control mice rise from pretreatment values below the detection limit to a mean value of 468±79 pg/ml serum. The pretreatment with CsA at a dose of 10 mg/kg orally reduces the INF-γ serum levels to 90%. At a dose of 20 mg/kg b.w., IFN-γ levels drops to 12%. A further reduction to about 7% is seen at a dose of 30 mg/kg. Increasing the CsA dose to 40 and 80 mg/kg b.w. results in IFN-γ levels of 2% and 4% respectively. The IL-4 serum levels in response to various doses of CsA are shown. IL-4 serum levels rise from non detectable values to a mean value of 342±71 pg/ml serum in the control mice. A CsA dose of 10 mg/kg b.w. reduces the serum IL-4 concentration to 65%. The dose of 20 mg/kg b.w. reduces the IL-4 levels in serum to 1.5% and at a dose of 30 mg/kg the IL-4 level is 10%. The CsA doses of 40 and 80 mg/ml has only 4% of the IL-4 measured in the control mice. The results of this experiment show that CsA administered orally at doses ranging from 10 to 80 mg/kg is very potent in reducing the antigen induced production of the cytokines IL-2, IFN-γ and IL-4 in DO11.10 mice. For IL-2 an ED₅₀ value of CsA of 14.3±1.4 mg/kg is calculated. For the calculation of an an ED₅₀ for IFN-γ and IL-4 additional doses have to be applied because of the steepness of the dose response curves.

### Example 3

### DO11.10 Mice with Priming and Triggering Agents

A) Experimental Setup

a) Animals

Animals are provided as described in Example 2a).

b) Administration of OVA₃₂₃₋₃₃₉ (=OVA-peptide) and LPS Groups of six DO11.10 mice receive i.p. 30 μg of OVA₃₂₃₋₃₃₉ peptide (98% purity, pi Chem R&D, Graz Austria) in 0.2 ml 0.9% saline per mouse. Control mice are injected i.p. with the same amount of saline.

18 hours later the mice are challenged i.p. with 100, 300 or 500 μg LPS (E. coli 0111: B4 Sigma Vienna, Austria). At 2, 4 and 6 hours after the LPS administration about 200 μl of blood are collected by puncture of the retro-orbital plexus under ether anesthesia. The sera are collected and stored frozen at −20° until analyzed.

c) Quantification of Cytokines

Murine IL-1α, IL-1β and TNF-α serum levels are determined by ELISAs (IL-1α and TNF-α, Bender MedSystems, Vienna Austria; IL-1β, Quantikine R&D Systems, Minneapolis, Minn.). Results are expressed as pg per ml serum. Differences in the serum cytokines concentrations between the different experimental groups are examined for statistical significance by the Mann-Whitney U test, two tailed. Results are expressed as the mean±SEM. p<0.05 is regarded as significant.

B) Results

1) IL-1α Serum Levels

The injection of OVA-peptide at doses between 3 and 90 μg per mouse increases serum concentrations of IFN-γ as well as other cytokines, such as IL-4 and IL-2, in a dose/amount-dependent way. Maximum concentrations, up to more then several 100 fold above pretreatment values, are reached 2 to 4 hours after i.p. administration of 30 or 90 μg of OVA-peptide. Since no difference is found in the serum IFN-γ levels between doses of 30 and 90 μg of OVA-peptide, a dose of 30 μg is chosen for the priming of the DO11.10 mice. Values for serum IL-1α responses in control DO11.10 mice challenged with various doses of LPS and in mice primed with the OVA-peptide 18 hours prior to challenge are given in TABLE 6. The serum IL-1α levels are expressed as mean pg per ml serum±SEM.

TABLE 6

| | IL-1α level in pg/ml serum after | | |
|---|---|---|---|
| administration of | 2 hours | 4 hours | 6 hours |
| NaCl + 100 μg LPS | 38.699 +/− 5.074 | 45.230 +/− 8.495 | 24.874 +/− 7.586 |
| NaCl + 300 μg LPS | 28.691 +/− 6.006 | 37.682 +/− 7.839 | 31.575 +/− 9.990 |
| NaCl + 500 μg LPS | 28.691 +/− 6.006 | 37.682 +/− 7.839 | 31.575 +/− 8.880 |
| OVA₃₂₃₋₃₃₉ + 100 μg LPS | 42.686 +/− 4.661 | 63.890 +/− 29.615 | 103.075 +/− 41.810 |
| OVA₃₂₃₋₃₃₉ + 300 μg LPS | 36.833 +/− 9.638 | 171.013 +/− 54.849 | 192.811 +/− 61.966 |
| OVA₃₂₃₋₃₃₉ + 500 μg LPS | 118.596 +/− 14.518 | 149.724 +/− 52.643 | 524.951 +/− 111.299 |

The treatment with LPS at doses of 100, 300 or 500 μg per mouse induces IL-1α serum levels in the range of 22.9±6.7 to 56.9±5.7 pg per ml serum. However, no dose dependency in the IL-1α serum levels can be detected in control mice when the sera are analyzed 2, 4 and 6 hours after the LPS challenge. OVA-peptide priming results in a rapid increase in IL-1α serum levels when challenged with the highest LPS dose.

2) IL-1β Serum Levels

Values of serum IL-1β responses in control DO11.10 mice challenged with various doses of LPS (=NaCl+LPS) and in mice primed with the OVA-peptide 18 hours prior to the LPS challenge (=OVA+LPS) are given in TABLE 7. The serum IL-1β levels are expressed as mean pg per ml serum±SEM.

TABLE 7

| | IL-1β level in pg/ml serum after | | |
|---|---|---|---|
| administration of | 2 hours | 4 hours | 6 hours |
| NaCl + 100 μg LPS | 252.303 +/− 68.712 | 295.269 +/− 35.911 | 153.009 +/− 73.919 |
| NaCl + 300 μg LPS | 169.221 +/− 128.303 | 355.218 +/− 24.992 | 554.143 +/− 83.078 |
| NaCl + 500 μg LPS | 148.578 +/− 8.674 | 558.418 +/− 30.431 | 680.661 +/− 57.453 |
| OVA₃₂₃₋₃₃₉ + 100 μg LPS | 426.984 +/− 72.930 | 512.215 +/− 247.248 | 1198.531 +/− 392.919 |
| OVA₃₂₃₋₃₃₉ + 300 μg LPS | 331.063 +/− 91.489 | 957.433 +/− 465.283 | 1712.753 +/− 655.367 |
| OVA₃₂₃₋₃₃₉ + 500 μg LPS | 517.428 +/− 110.161 | 1223.519 +/− 475.491 | 3866.987 +/− 696.588 |

The IL-1β serum levels in unprimed mice challenged with 100 μg of LPS are 252.3±68.7 pg/ml 2 hours after the challenge. The corresponding concentration in OVA-peptide primed mice is 1.6 fold higher. 4 hours after the LPS challenge, the concentrations are similar with a 1.7 fold increase. After 6 hours the values are 7.8 fold increased in OVA-peptide primed animals. The challenge of unprimed mice with 300 μg of LPS results in a mean concentration of 169.2±18.3 pg/ml and a 1.9 fold increase is found in the OVA-peptide primed mice after 2 hours. After 4 hours the IL-1β concentration in the OVA-peptide primed mice rises 2.7 fold as compared to an unprimed mean. 6 hours after the LPS challenge, a 3.0 fold increase can be measured in the OVA-peptide primed animals. The LPS challenge with a dose of 500 μg per mouse induces a 3.5 fold rise in the OVA-peptide primed mice after 2 hours. After 4 hours IL-1β levels are 2.2 fold higher. A substantial rise of a 5.7 fold increase in the IL-1β levels can be detected in OVA-peptide primed mice 6 hours after the LPS challenge.

3) TNF-α Serum Levels

The effects of various doses of LPS (100, 300, or 500 μg/mouse) on the production of TNF-α in the serum of non-sensitized DO11.10 mice or mice sensitized by i.p. injection of OVA-peptide (30 μg/mouse, 18 hours prior to LPS challenge) are given in TABLE 8. The TNF-α serum levels are expressed as mean pg per ml serum±SEM.

TABLE 8

| | TNF-α level in pg/ml serum after | | |
|---|---|---|---|
| administration of | 2 hours | 4 hours | 6 hours |
| NaCl + 100 μg LPS | 672.286 +/− 619.156 | 95.660 +/− 24.845 | 188.909 +/− 169.018 |
| NaCl + 300 μg LPS | 1144.708 +/− 286.542 | 567.901 +/− 432.819 | 39.641 +/− 11.835 |
| NaCl + 500 μg LPS | 1505.555 +/− 204.078 | 162.556 +/− 34.477 | 241.637 +/− 142.935 |
| OVA₃₂₃₋₃₃₉ + 100 μg LPS | 6552.911 +/− 2645.343 | 133.059 +/− 53.186 | 105.297 +/− 37.387 |
| OVA₃₂₃₋₃₃₉ + 300 μg LPS | 8979.088 +/− 2907.689 | 385.924 +/− 194.462 | 253.396 +/− 136.190 |
| OVA₃₂₃₋₃₃₉ + 500 μg LPS | 7891.200 +/− 2394.159 | 222.733 +/− 104.548 | 321.129 +/− 57.176 |

2 hours after the challenge with 100 μg of LPS, the TNF-α levels are 672±61.9 pg/ml in unprimed animals and 9.7 fold higher in OVA-primed mice. At a challenge dose of 300 μg of LPS, a mean of 1144.708±286.5 pg/ml can be determined in the sera of unprimed animals. The OVA-peptide priming increases the TNF-α concentration 7.8 fold. OVA-peptide priming before challenged with 500 μg of LPS increases the amount 5.2 fold. 4 hours after the challenge with 100 μg of LPS, the TNF-α levels decline to 95.6±24.8 pg/ml in unprimed mice. In OVA-primed animals no significant changes in the TNF-α levels can be determined (133.0±53.1 pg/ml). A similar effect of no difference in the TNF-α levels between unprimed and OVA-peptide primed animals is seen at LPS challenge doses of 300 μg.

6 hours after the LPS challenge, the TNF-α concentrations in the sera of the OVA-primed mice are 105.2±37.3 pg/ml at 100 μg of LPS; 253.4±136.2 pg/ml at 300 μg of LPS and 321.1±57.1 pg/ml at 500 μg of LPS.

1. A kit for the determination of a T-cell and/or inflammatory effector cell derived mediator directly in vivo in serum, comprising a mouse wherein the majority of T cells express a transgenic MHC class I restricted or MHC class II restricted T cell receptor.

2. A kit according to claim 1 comprising:
a. a mouse wherein the majority of T cells express a transgenic MHC class I restricted or MHC class II restricted T cell receptor;
b. a MHC restricted TCR specific OVA-peptide;
c. optionally a triggering agent;
d. optionally controls, standards and/or calibration means;
e. optionally detection means for a T-cell and/or inflammatory effector cell derived mediator; and
f. instructions for using the components of said kit.

3. A process for determination of a T-cell and/or inflammatory effector cell derived mediator in serum of an OVA-peptide stimulated mouse wherein the majority of T cells express a transgenic MHC class I or MHC class II restricted T cell receptor, said process comprises the steps of:
a. administering to a mouse wherein the majority of T cells express a transgenic MHC class I restricted or MHC class II restricted T cell receptor a MHC restricted TCR specific OVA-peptide and optionally a triggering agent; and
b. determining the level of a T-cell and/or inflammatory effector cell derived mediator produced.

4. A method for identifying an agent that interferes with T cell activation and/or -differentiation and/or modulation of other inflammatory effector cells comprising the steps of:
a. administering to a mouse wherein the majority of T cells express a transgenic MHC class I or MHC class II restricted T cell receptor a MHC restricted TCR specific OVA-peptide and optionally a triggering agent;
b. administering to a transgenic mouse of step a) a candidate compound before, after or simultaneously with the peptide and optionally a triggering agent of step a);
c. determining the level of a T-cell and/or inflammatory effector cell derived mediator in serum of
c1. a mouse treated according to step a), and
c2. a mouse treated according to step b) and step a),
d. determining whether there is a difference in the level of said mediator produced in said serum; and
e. choosing an agent as determined in step d).

5. The use of an agent identified by a method of claim 4 as a pharmaceutical.

6. The use of a mouse wherein the majority of T cells express a transgenic MHC class I or MHC class II restricted T cell receptor in a method for the identification of an agent that interferes with T cell activation and/or -differentiation and/or modulation of other inflammatory effector cells.

7. The use of a mouse wherein the majority of T cells express a transgenic MHC class I or MHC class II restricted T cell receptor in a method for the determination of a T-cell and/or inflammatory effector cell derived mediator in the serum of said mouse.

8. A pharmaceutical composition comprising at least one agent that interferes with T cell activation and/or -differentiation and/or modulation of other inflammatory effector cells identified by the method of claim 4 beside pharmaceutically acceptable excipient(s).

9. A method for the treatment of a disease which is based on an unwanted or aberrant immune response, comprising administering an agent identified by a method of claim 4 or a pharmaceutical composition of claim 8 to a subject in need of such a treatment.

10. A method according to claim 9 characterized in that the disease which is based on an unwanted or aberrant immune response is selected from the group consisting of allergic disease, transplantation, autoimmune related disease, inflammatory disease and modulation/stimulation of a tumor specific or pathogen specific response.
